(19) United States
(12) Patent Application Publication
(10) Pub. No.: US 2024/0325173 A1
(43) Pub. Date: Oct. 3, 2024

(54) Adaptive Compressive Prosthetic Device and Methods Of Using The Same

(71) Applicant: Joe JOHNSON, Windsor, CO (US)

(72) Inventor: Joe JOHNSON, Windsor, CO (US)

(21) Appl. No.: 18/578,602

(22) PCT Filed: Jul. 16, 2022
(86) PCT No.: PCT/US2022/037401

§ 371 Date: Jan. 11, 2024

Related U.S. Application Data
(60) Provisional application No. 63/222,897, filed on Jul. 16, 2021.

(51) Int. Cl. A61F 2/78; A61F 2/60

(52) U.S. Cl. CPC A61F 2/7812; A61F 2/604

(57) ABSTRACT

The inventive technology disclosed herein relates to a novel compression cell device and methods of use thereof comprises: an adaptable compression prosthetic device that may be configured to adaptably secure a residual limb using one or more of compression cells, that may further incorporate a thermal indicator system to identify areas of high and low residual limb engagement with the cells: adaptable compression socket that may be configured to adaptably secure a residual limb using one or more of said novel compression cells that may be further secured in a compression slot and secured by one or more compression cords that is responsive to an compression actuator: said adaptable compression socket that may be further include a thermal surface.

### CROSS-REFERENCE TO RELATED APPLICATIONS

This International PCT application claims the benefit of and priority to U.S. Provisional Application No. 63/222,897 filed Jul. 16, 2021. The specification, claims and drawings of which are incorporated herein by reference in their entirety.

### TECHNICAL FIELD

Generally, the inventive technology disclosed herein relates to the field of prosthetic devices, and more particularly an adaptable compression prosthetic device that may be configured to adaptably secure a residual limb using one or more compression cells.

### BACKGROUND OF THE INVENTION

Traditional prosthetic devices operate by securing the residual limb into a rigid or semi-rigid socket. A socket may commonly refer to the portion of a prosthesis that fits around and secures a residual limb, and to which prosthetic components, such as a foot, are attached. Traditional prosthetic devices are generally designed to stabilize the skeletal components of the residual limb and allow minimal relative movement between the socket and the residual limb. To effectively support the residual limb and allow for the efficient transfer loads from the residual limb to the ground, traditional prosthetic sockets are designed to provide sufficient support to secure the residual limb within the socket, while at the same time allowing sufficient flexibility to allow for circulation and account for other physiological, temporal or environmental changes that may affect the shape and/or volume of the residual limb.

One significant drawback of traditional prosthetic sockets is the inability to account for shape and volume fluctuations of the residual limb. Traditional prosthetic sockets are generally produced in a fixed or static form such that they do not have the ability to accommodate changes in the residual limb-socket interface. For example, it is known that a number of factors may cause a residual limb to change shape and or present an altered volumetric profile. Shape and volume fluctuations in a residual limb may be due to many factors, including but not limited to: edema, muscle atrophy, weight gain/loss, renal dialysis, salt or water intake, alcohol consumption, menses, and changes in wearing time and activity. Additionally, the act of wearing a prosthetic socket, in combination with the mechanical action of walking, or other movements causes a reduction in the overall volume of a residual limb over time. When there are shape and volume fluctuations, the residual limb-socket interface is compromised, which can lead to discomfort, pain, destabilizing motion between the socket and residual limb, as well as damage to surrounding soft tissue.

As such, there exists a need for a prosthetic socket that may overcome the limitations of prior traditional systems. The current invention overcomes the limitations of, and indeed surpasses the functionality of traditional prosthetic socket systems. It is therefore the object of the present invention to provide a simple, versatile, cost effective, prosthetic socket system that can be fully-adaptable to a user's needs. Specifically, one aim of the present technology is to provide a plurality of individual compression cells that can be coupled with a soft inner prosthetic liner, rigid prosthetic socket frame or a combination of the same to provide an adaptable compression force on the residual limb within the body of the socket based on a user's need.

The present invention further provides for prosthetic socket that allows for anatomically directed compression, as well as features to accommodate soft tissue expansion through the application of a plurality of compression cells configured to be coupled with a prosthetic socket frame. These compression cells may be responsive to a compression actuator that is configured to exert a lateral compression force on a residual limb within the socket.

### SUMMARY OF INVENTION

One object of the inventive technology disclosed herein includes a novel compression cell device methods of use thereof.

Another object of the inventive technology disclosed herein includes adaptable compression prosthetic device that may be configured to adaptably secure a residual limb using one or more of said novel compression cells.

Another object of the inventive technology disclosed herein includes adaptable compression liner that may be configured to adaptably secure a residual limb using one or more of said novel compression cells.

Another object of the inventive technology disclosed herein includes adaptable compression liner that may be configured to adaptably secure a residual limb using one or more of said novel compression cells.

Another object of the inventive technology disclosed herein includes adaptable compression liner that may be configured to adaptably secure a residual limb using one or more of said novel compression cells secured to the internal frame surface of the socket.

Another object of the inventive technology disclosed herein includes adaptable compression socket that may be configured to adaptably secure a residual limb using one or more of said novel compression cells that may be further secured in a compression slot and secured by one or more compression cords that is responsive to an compression actuator.

Another object of the inventive technology disclosed herein includes adaptable compression socket that may be configured to adaptably secure a residual limb using one or more of said novel compression cells that may be further include a thermal surface.

Further objects of the inventive technology will become apparent from the description and drawings below.

### BRIEF DESCRIPTION OF THE DRAWINGS

The inventive disarticulated compression socket described with reference to the accompanying drawings which show preferred embodiments according to the device described herein. It will be noted that the device as disclosed in the accompanying drawings is illustrated by way of example only. The various elements and combinations of elements described below and illustrated in the drawings can be arranged and organized differently to result in embodiments which are still within the spirit and scope of the device described herein.

FIG. 1A-D: shows multiple view of a compression cell in one embodiment thereof;

FIG. 2A-D: (A-C) shows a plurality of compression cells coupled to the internal and external surfaces of with a prosthetic liner; (D) shows a plurality of compression cells coupled to the external frame surface of an exemplary socket in one embodiment thereof;

FIG. 3A-E: shows multiple view of a compression cell in an alternative embodiment coupled with an adaptor support having a plurality of insert channels in one embodiment thereof;

FIG. 4A-C: shows multiple view of a disarticulated compression cell in an alternative embodiment with an adaptor support having a plurality of insert channels in one embodiment thereof;

FIG. 5: shown a prosthetic liner having a plurality of cell interfaces securing a compression cell positioned within a socket frame further having a plurality of disarticulated compression cell coupled with the socket frame and configured to provide an inward compressive force when actuated in one embodiment thereof;

FIG. 6A-D: shown a plurality of compression cells configured to be coupled with a plurality of adaptor supports and optional linkers forming a pair of lateral insert channels in one embodiment thereof;

FIG. 7A-F: show an exemplary prosthetic socket frame configured to secure a plurality of compression cells configured to be coupled with a plurality of adaptor supports and optional linkers within a plurality of compression slots and further be responsive to one or more compression cords, which is further responsive to an actuator, and configured to exert a lateral compression force on the coupled compression cells on a residual limb within the socket; and

FIG. 8A-F: show an exemplary disarticulated prosthetic socket frame configured to secure a plurality of compression cells configured to be coupled with a plurality of adaptor supports and optional linkers within a plurality of compression slots and further be responsive to one or more compression cords, which is further responsive to an actuator, and configured to exert a lateral compression force on the coupled compression cells on a residual limb within the socket.

### DETAILED DESCRIPTION OF THE INVENTION

The present invention includes a variety of aspects, which may be combined in different ways. The following descriptions are provided to list elements and describe some of the embodiments of the present invention. These elements are listed with initial embodiments; however, it should be understood that they may be combined in any manner and in any number to create additional embodiments. The variously described examples and preferred embodiments should not be construed to limit the present invention to only the explicitly described systems, techniques, and applications. Further, this description should be understood to support and encompass descriptions and claims of all the various embodiments, systems, techniques, methods, devices, and applications with any number of the disclosed elements, with each element alone, and also with any and all various permutations and combinations of all elements in this or any subsequent application.

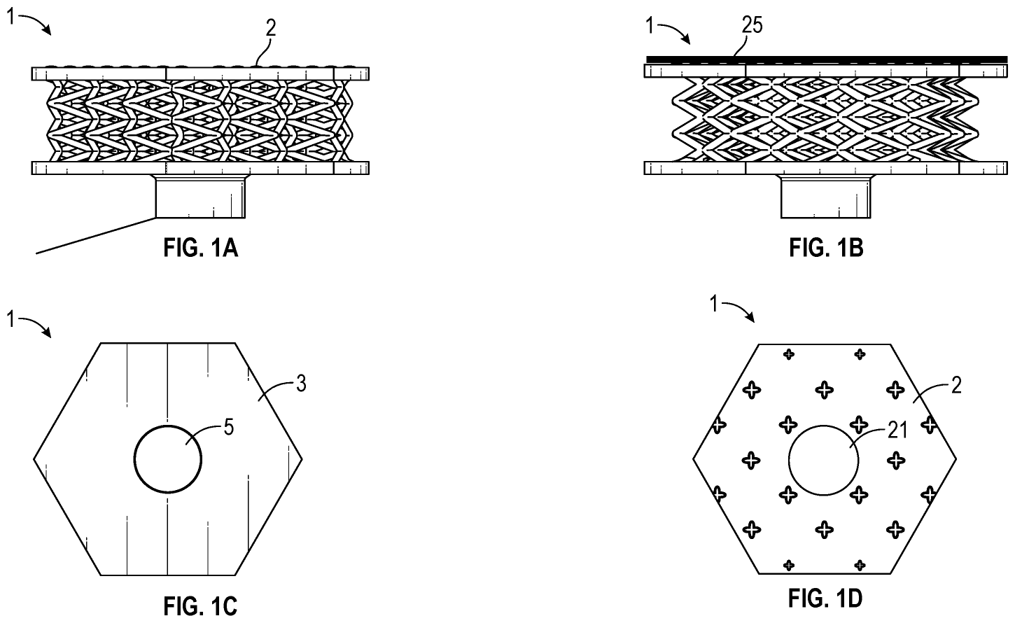

The inventive technology may include a compression cell (1), which as shown in FIGS. 1A-D, may include, in one embodiment, a compression matrix (4) positioned between a contact surface (2) and a support surface (3), and may further optionally include one or more adaptors (5) positioned on the support surface (3). In this preferred embodiment, the compression matrix (4) may be configured to be compressible in response to a force applied to the contact, or support surfaces (2,3). The width and material of the compression matrix (4) can be variable to accommodate a variety of applications. For example, as generally shown in FIGS. 1A-B, in one embodiment, the compression matrix (4) of the invention may include a collapsible scaffold matrix configured to compresses in response to an externally applied force. In this embodiment, the collapsible scaffold matrix is formed by a plurality of interconnected series of cantilevered supports that are configured to be compressed in response to an externally applied force. As noted below, the compression matrix (4) of the compression cell (1) may be formed of a material having sufficient elasticity/rigidity to allow it to be compressed in response to an external force and decompressed and return to its original configuration once that force has been removed. As noted below, the application of a collapsible scaffold matrix allows for the use of rigid materials for the contact, or support surfaces (2,3) and adapter (5) while maintaining sufficient elasticity in the compression matrix (4).

Notably, a variety of matrix configurations or materials may be contemplated in this invention. For example, in certain alternative embodiments, the compression matrix (4) may include a solid compressible material, such as a gel or soft plastic or other elastic material. In additional embodiments, a collapsible scaffold matrix may include a series of springs or other supports that are positioned between the contact and support surfaces (2,3) respectively, and further configured to be compressed in response to an externally applied force, such as applied by contact with a residual limb in one embodiment.

Again, referring to FIGS. 1A-D, a compression matrix (4), contact surface (2), and support surface (3) as well as optionally the one or more adaptors (5) positioned on the support surface (3) may comprise a single integral component. In this preferred embodiment, the integral compression cell (1) may be formed by a 3D-printing process, however additional methods of manufacture are also contemplated within the scope of the invention, including various molding techniques, such an injection, compression, blow, or rotational molding and the like.

One or more compression cells (1) of the invention may be configured to be secured to a prosthetic liner (6), a prosthetic socket frame (10), or a combination of the same. In this embodiment, one or a plurality of compression cells (1) of the invention may be positioned so as to be placed adjacent to the residual limb within the prosthetic liner (6) of socket frame (10) and provide a compressive support structure. The position, size, and shape of the individual compression cells (1) in relation to the secured residual limb within the prosthetic device, as well as the elasticity and or rigidity of the compression matrix allows a user to strategically position one, or a plurality of compression cells (1) to generate an adaptive compression profile on the residual limb.

As used herein, the term “adaptive compression profile” means the placement of one or a plurality of compression cells (1) adjacent to a residual limb secured to a portion of a prosthetic device such that the compression cell(s) (1) provide a customizable compression support profile against the residual limb. As noted above, this adaptive compression profile can be adjusted to fit the shape or placement of a residual limb within a prosthetic device and may further be adjusted to accommodate changes in the size and shape of the residual limb that often occur in response to environmental factors, or during certain activities. In this manner, a user may continually adapt the position, size, and type of the compression cells (1) in relation to the residual limb to generate a fully-customizable adaptable compression profile.

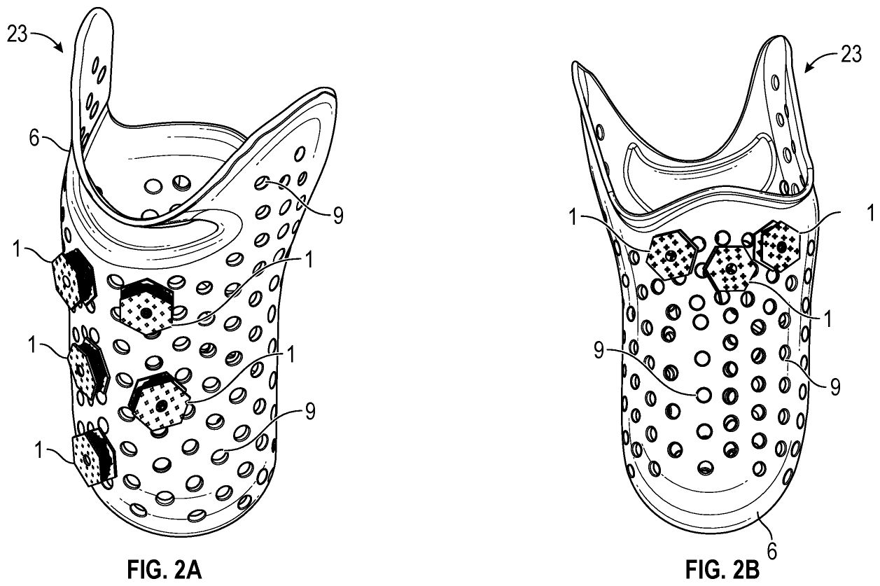

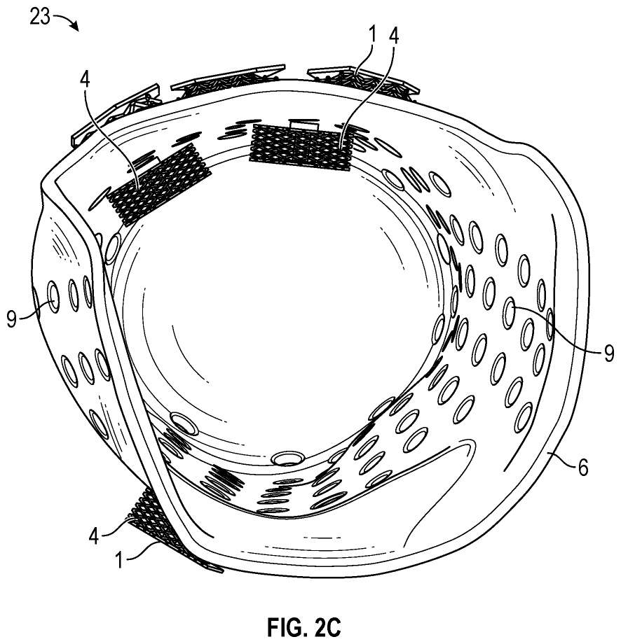

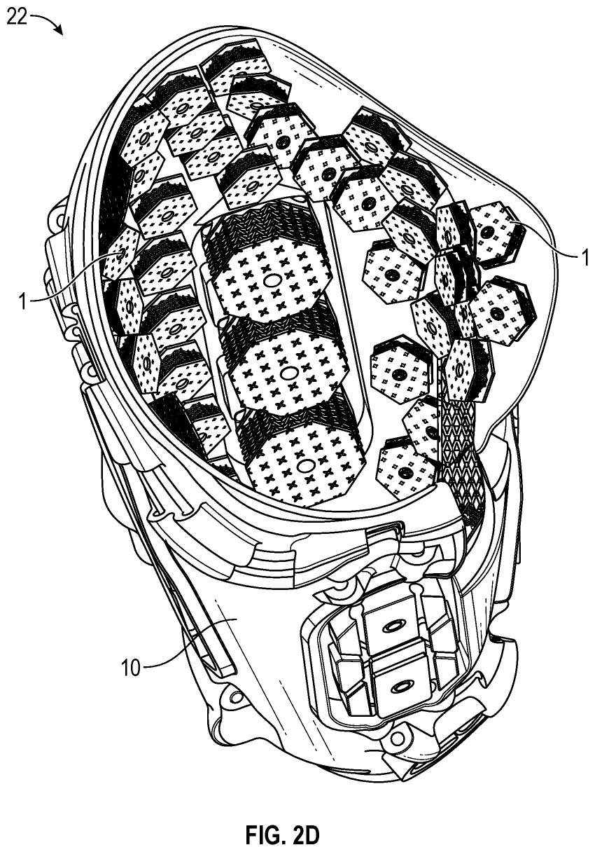

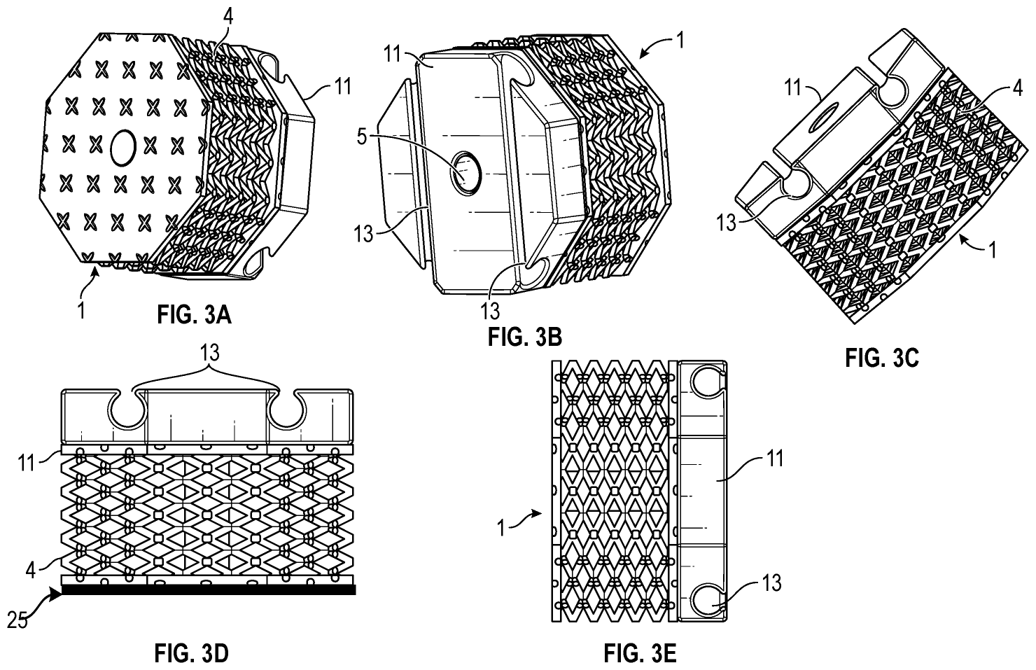

In one preferred embodiment, an adaptable compression support profile may be generated by securing one, or a plurality of compression cells (1) to prosthetic liner (6) having a plurality of cell interface (9) positions forming an adaptive compression liner (23). As shown in FIGS. 2A-D and 5, a prosthetic liner (6) may include a plurality of cell interface (9) positions that can be further configured to secure a compression cell (1). In this embodiment, the adaptor (5) of a compression cell (1) may be secured with the cell interface (9) of the liner (6).

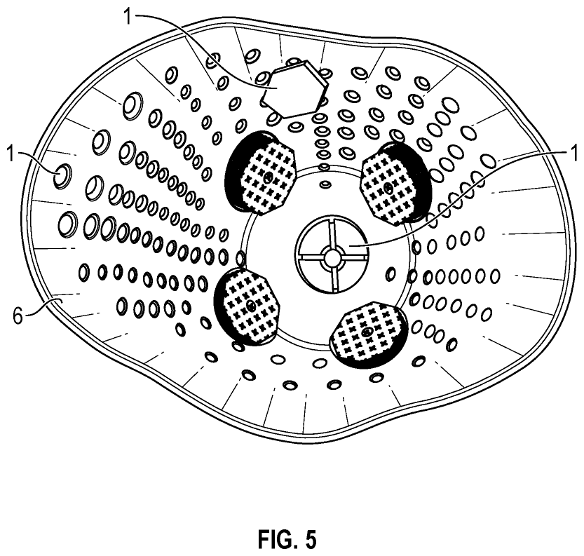

As further shown in FIGS. 2 and 5, the adaptor (5) of a compression cells (1) may be secured within a cell interface (9) such that the compression matrix (4) of the cell (1) projects from the inner surface (7) or external surface (8) of the liner, respectively. In this configuration, a user may secure one or more compression cells (1) to the inner surface (7) of the liner such that the compression matrix (4) is positioned adjacent to the residual limb with the contact surface of the cell (1) being in contact with the limb providing a compressive support against the limb when secured within a rigid socket frame (10). Alternatively, or in conjunction with the aforementioned embodiment, a user may secure one or more compression cells (1) to the external surface (8) of the liner such that the compression matrix (4) is positioned adjacent to the rigid socket frame (10) with the contact surface of the cell (1) being in contact with the internal frame surface (24) of the socket frame (10) thereby providing a compressive support against the prosthetic liner (6), and the limb disposed of therewith, against the internal frame surface (24) of the rigid socket frame (10).

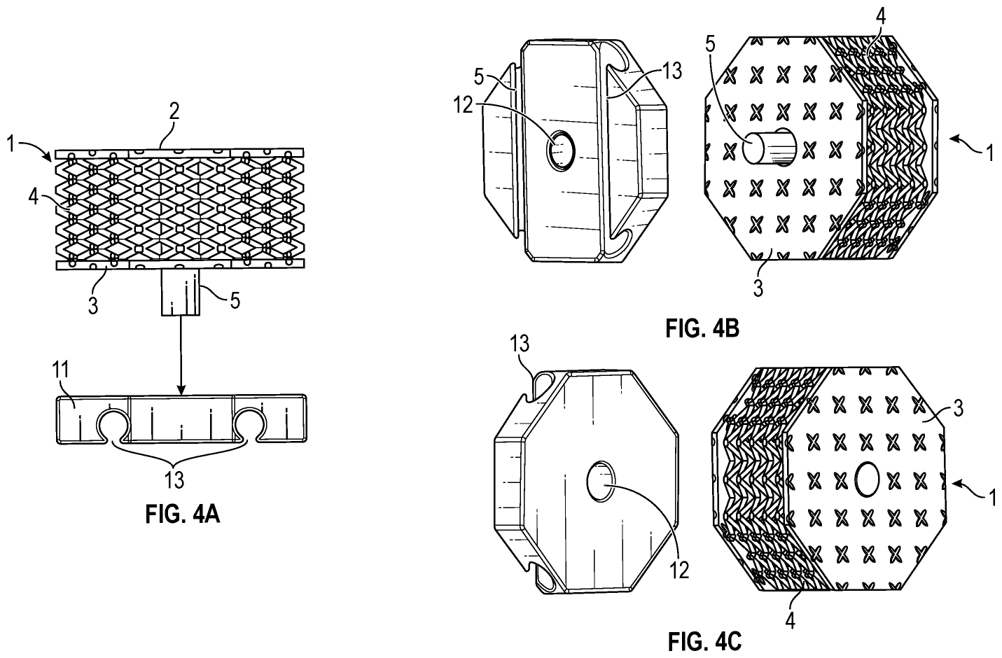

In another preferred embodiment, an adaptable compression support profile may be generated by securing one, or a plurality of compression cells (1) to the internal frame surface (24) of a socket frame (10) having a plurality of cell interface (9) positions. As shown in FIG. 4D, a socket frame (10) may include a plurality of cell interface (9) positions that can be further configured to secure a compression cell (1). In this embodiment, the adaptor (5) of a compression cell (1) may be secured within the cell interface (9) on the socket frame (10). As further showing in FIG. 5B, the adaptor (5) of a compression cells (1) may be secured with a cell interface (9) positioned on the internal frame surface (24) of the socket frame (10) such that the compression matrix (4) of the cell (1) is projecting into the residual limb cavity of the socket frame (10).

In this configuration, or in conjunction with the aforementioned embodiments described above, a user may secure one or more compression cells (1) to the internal frame surface (24) of the socket frame (10) such that the compression matrix (4) extends from the the rigid socket frame (10), In this configuration, the contact surface of the cell (1) is in contact with the external surface (8) of the prosthetic liner (6) thereby providing a compressive support against the prosthetic liner (6), and the limb disposed of therewith, against the internal frame surface (24) of the rigid socket frame (10).

While the preferred embodiment shown in FIGS. 1-2 demonstrates the adaptor (5) being a solid extension that may be secured with an aperture cell interface (9), a variety of adaptor (5) and cell interface (9) configurations are contemplated within the invention. For example, in one embodiment, the adaptor (5) of the invention may include a coupler configured to be secured to a cell interface (9), or other external surface. In this embodiment, a coupler configured to be secured to an external surface may comprise a coupler selected from the group consisting of: a snap coupler, a slide coupler, a suction coupler, a twist coupler, a lock, or a combination of the same. Additional embodiments may include one or more features to secure the adaptor (5) to the cell interface (9) such as an extended catch surface, such as a bevel or other similar extended component.

Now referring to FIG. 1, a compression cell (1) of the invention my include a secondary adapter (21) positioned on the cell's contact surface (2). In this embodiment, a second compression cell (1) may be coupled with a first compression cell (1) by coupling the adaptor (5) of the second cell (1) with the secondary adapter (21) of the first cell (1). In this configuration, a user can modify the number and position of a plurality of compression cells (1) in relation to the residual limb, prosthetic liner (6), or socket frame (10) to generate an adaptable compression profile.

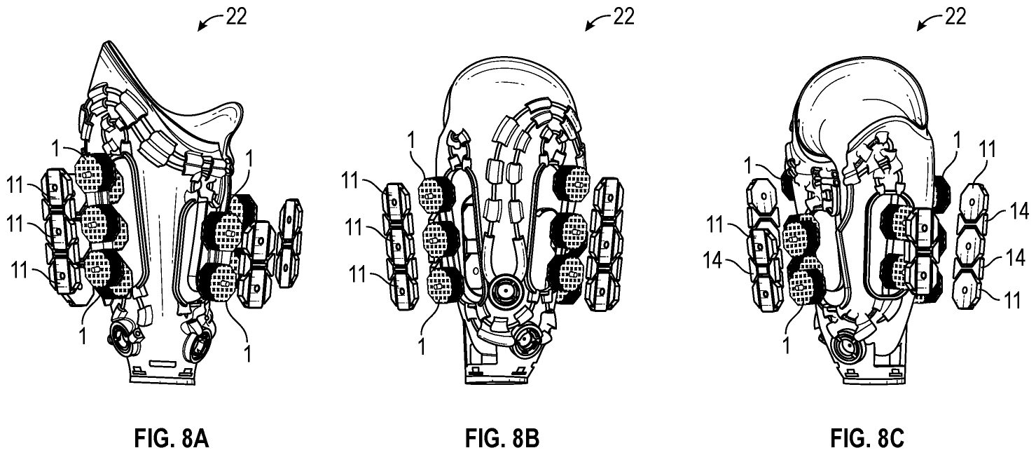

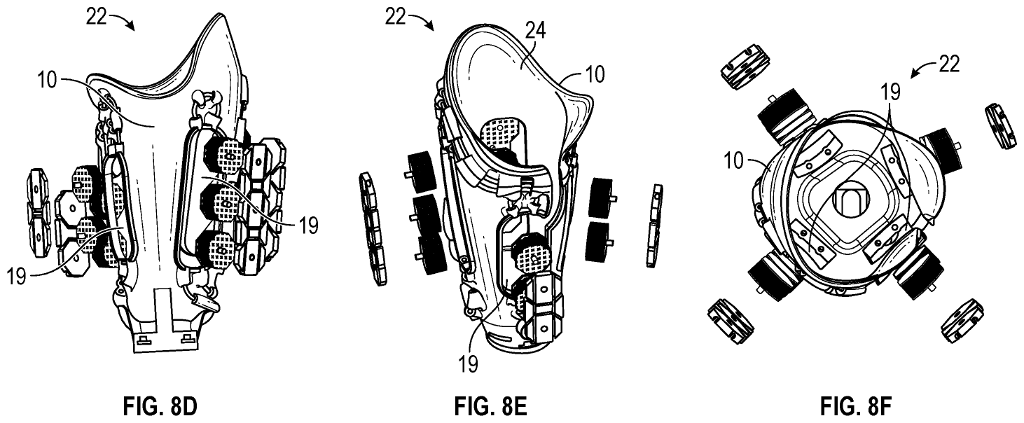

Generally referring to FIGS. 8-10, the inventive technology may include an adaptive compression socket (22). In one embodiment, an adaptive compression socket (22) may include a socket frame (10). As part of the inventive technology, such socket frames (10) may be custom formed to accommodate the anatomical shape of a specific user's residual limb. Considerations of shape, weight distribution, volume and movement may all be considered in forming the socket frame's (10) overall shape and configuration. A socket frame (10) may also be coupled with a joint coupler (20) that may be configured to secure additional prosthetic components such as artificial limbs, mechanical devices, as well as shock absorbers and the like.

A socket frame (10) may be a rigid form configured to accommodate a residual limb (not shown). One exemplary embodiment may include a transfemoral socket frame, or a socket frame (10) configured to accommodate a residual limb wherein the knee joint has been removed and the individual still has part of the femur or thighbone intact. Additional embodiments not specifically shown may also be contemplated, which may include, but not limited to: a transhumeral socket frame; a transradial socket frame; a transtibial socket frame; a symes socket frame; a hip disarticulation socket frame; a knee disarticulation socket frame; and a wrist disarticulation socket frame and the like. As noted above, in a preferred embodiment a socket frame (10) may be formed of a rigid material to accommodate and provide a support for a residual limb. For example, a rigid socket frame (10) may be formed from a variety of materials, including but not limited to: plastic, composites, carbon fiber or even an acrylic laminate socket frame with a stiffening component such as carbon fiber and/or para-aremid synthetic fiber. In a preferred embodiment, a socket frame (10) may be generated by a 3D-printing device.

A socket frame (2) can be configured to secure an inner socket or prosthetic liner (6). In a preferred embodiment, an prosthetic liner (6) may be configured to be secured over a residual limb, and as noted above may include an adaptive compression liner (23) configured to secure one or more compression cells (1) to its inner or external surface (6,7). In a preferred embodiment, this prosthetic liner (6) may be made of a soft, and/or compressible material that may provide a buffer from a rigid socket frame (2). In certain embodiments, this prosthetic liner (6) may be made from a variety of materials, such as plastics, various thermoplastics, rubber, gel, mesh, and silicone as well as various appropriate compressible materials known in the art. In a preferred embodiment, a prosthetic liner (6), and in particular an adaptive compression liner (23) of the invention may include 3D-printed liner.

As shown in FIGS. 2, 5 and 8-10, the prosthetic liner (6) may include a residual limb interface configured to conform to the specific anatomical shape of a user's residual limb and may further have an outer surface configured to be secured within the interior of the socket frame (10). In one preferred embodiment the residual limb interface, generally encompassing the internal socket frame surface (24), may be configured to be closely mated with the user's residual limb providing support and shock adsorption, while the outer surface is configured to be mated with the socket interface on the user's socket frame (10) forming a fitted coupling. As noted above, one or more compression cells (1) of the invention may be positioned between the residual limb and prosthetic liner (6), or between the prosthetic liner (6) and the internal socket frame surface (24) forming a customizable compression system capable be generating an adaptable compression profile on a residual limb. While initially described as disparate components, in certain embodiments both the socket frame (10) and prosthetic liner (6) may be an integral component.

As shown in FIGS. 8-10, one embodiment the adaptive compression socket (22) of the invention may be coupled with one, or even a plurality of disarticulated compression cells (1) of the invention. For example, in the preferred embodiment shown in the figures, one or more compression cells (1) may be positioned within a compression slot (19), wherein the cells (1) may be configured to be sufficiently flexible so as to allow expansion and compression as herein described.

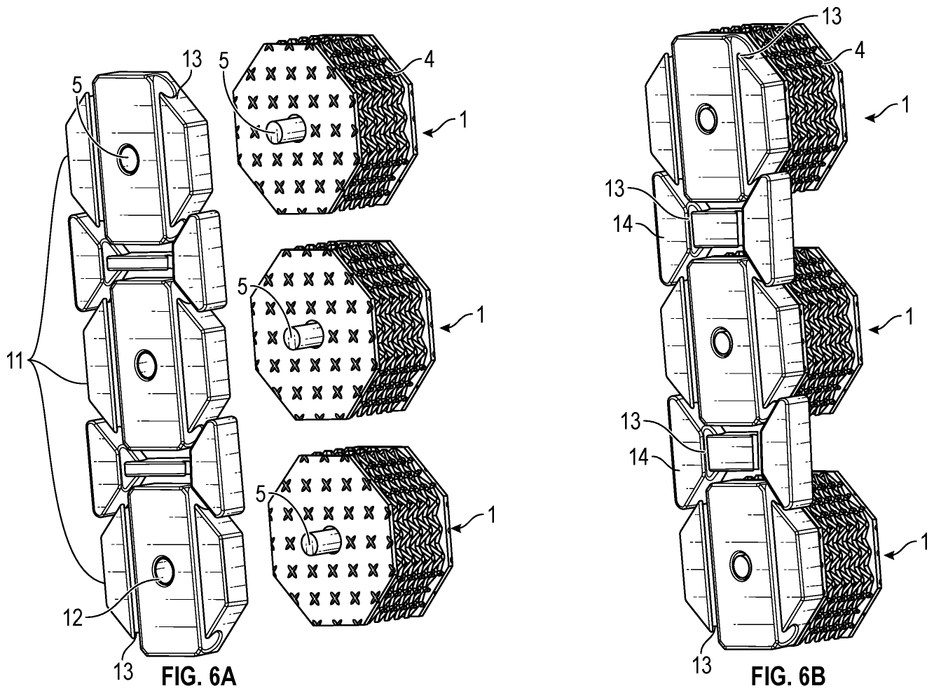

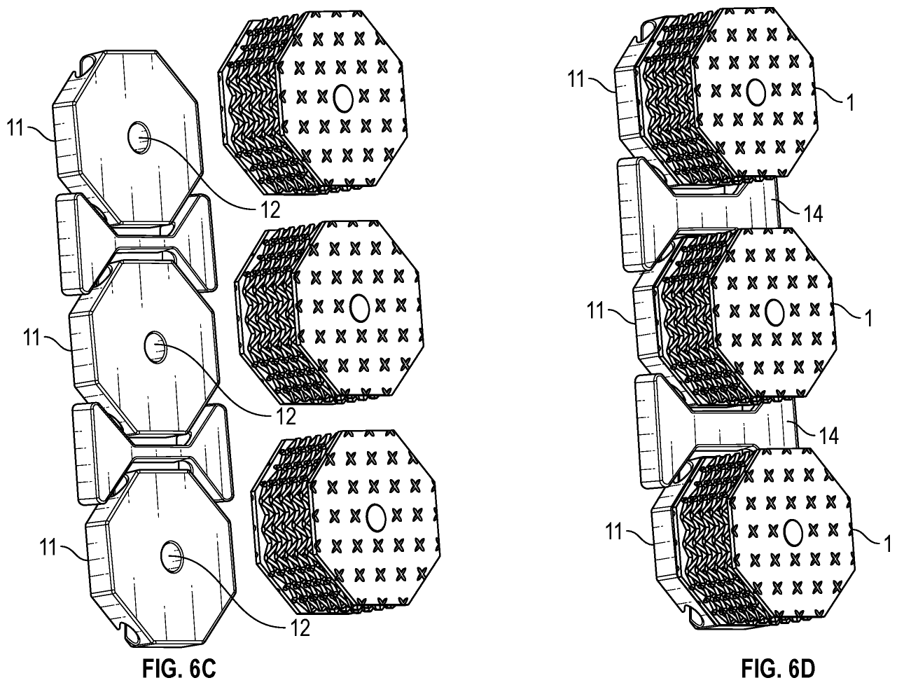

Again, referring to FIGS. 8-10, one, or a plurality of compression cells (1) may be positioned within a compression slot (19) and further secured in position by one or more compression cords (15) which may be responsive to a compression actuator (18). As shown in FIG. 6, a compression cell (1) of the invention may be coupled with an adaptor support (11) having one or more insert channels (13) configured to secure one or more compression cords (15). Notably, while this embodiment shows the compression cell (1) and adaptor support (11) as disparate elements, in certain embodiments they may form an integral component.

The described above, a compression cell (1) of the invention may include an extended compression matrix (4) terminating in a distal contact surface (3) configured to interface with the residual limb and/or prosthetic liner (3). Again, in this embodiment the compression matrix (4) may be formed from a compressible material that extends past the surface plane of the socket frame (10) and may form a cushioned interface with the residual limb or prosthetic liner (6).

In another embodiment, the invention may include one or more compression cells (1) each positioned within a compression slot (4) and further responsive to a compression actuator (18) through one or more compression cords (15). In a preferred embodiment, a compression cell (1) may include an extended compression matrix (4) that may be between 1/8 and 4 inches in thickness. In this embodiment, innervation of the compression actuator (7) may cause retraction of a compression cord (15) which causes the coupled compression cells (2), having an extended compression matrix (4), to contract generating an inward compressive force. This compressive force may work to secure a residual limb within socket frame (10). In this preferred embodiment, one or more compression cords (15) may be configured to be positioned within a cord surface channel (16) within the socket frame (10). Such a surface channel (16) may include a hollow aperture where a cord may be positioned such that it may be extended and/or retracted in response to a compression actuator (18). In this embodiment, one or more portions of the compression cord (15) may be anchored or represent an anchor cord position-such components being, in some cases the same. The compression cord (15) of the invention may be coupled with a compression actuator (18) and further positioned within a cord channel that traverses the socket frame (10) and at least one compression cell (1).

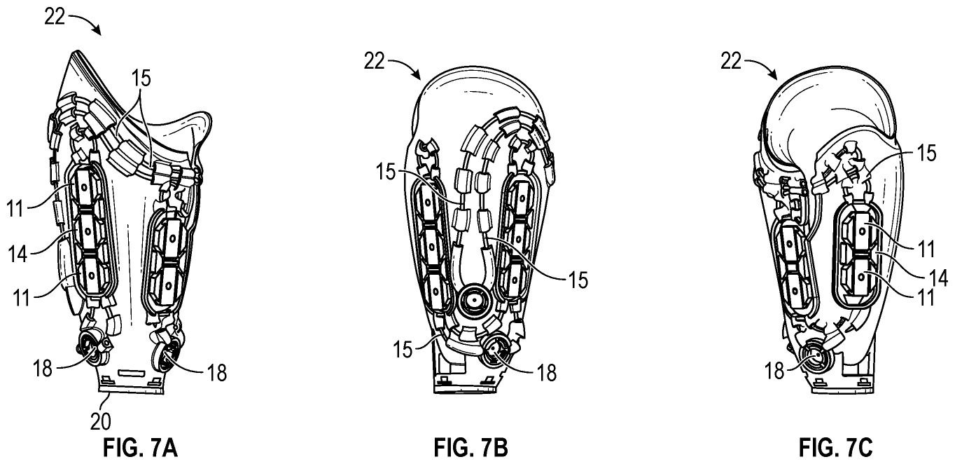

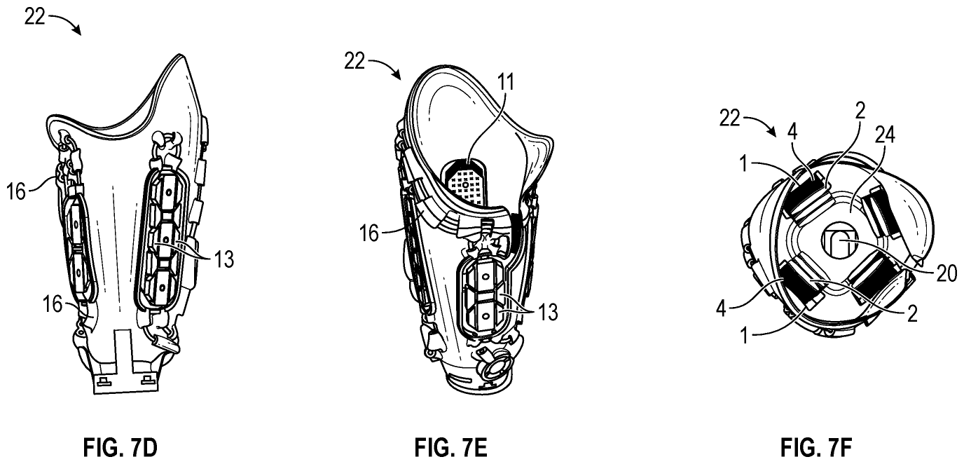

In still other embodiments, a compression cord (15) may be coupled with a compression actuator (18) and further positioned within a surface channel (16) that extends horizontally or laterally across the disarticulated compression cell (1) having an extended compression matrix (4). As show in FIG. 6, a compression cell (1) may be coupled with an adapter support (11) through an adaptor interface (12). This adapter support (11) may include a plurality of insert channels (13) that can secure a compression cord (15) responsive to a compression actuator (18). Moreover, as shown in FIGS. 6-7, in another embodiment, a plurality of compression cells (1) may each be coupled with an adapter support (11) through an adaptor interface (12) and further positioned in series such that the insert channels (13) are aligned, and preferably positioned parallel to the length of the body of the socket. As also shown in the figures, a linker (14) having one or more insert channels (13) may be positioned between two compression cells (1) placed in series such that all of the insert channels (13) are aligned. In this specific embodiment, a compression cord (15) may be secured within the series of aligned insert channels (13), such that when the compression actuator (18) is engaged, the compression cord (15) may be retracted or constricted. Here, because the extended compression matrix (4) causes the compression cell (1) to extend past the surface of the socket frame (10), the retraction of the compression cord (15) may allow the cell (1) to compress in a manner similar to the lateral compression described herein as the retracting cord is not retracting against the rigid frame of the socket frame (10), but the tractable surface of the compression matrix (4) of the cell (1).

In the preferred embodiment shown in the figures, a compression actuator (18) may be coupled with a compression cell (1) in such a manner as to secure it within the compression slot (19). This compression actuator (18) may further be configured to position and/or secure the compression cell (1) such that it is freely tractable in one, or multiple directions in response to the action of the actuator (18). It should be understood that for purposes of this invention a compression actuator (18) encompasses any apparatus that may be configured to adjust the movement of another portion of a compression cell (1). In a preferred embodiment, a compression actuator may be any apparatus that may be configured to adjust the movement of the socket frame (10), and/or a compression actuator (18) may be any apparatus that may be configured to adjust the movement of both. Examples of such compression actuators may include a strap compression actuator; an air pressure compression actuator; an automatic compression actuator; a twist compression actuator; and a detachable compression actuator.

As highlighted in FIGS. 5-10, a compression actuator (18) may be coupled with the compression cell (1) through one or a plurality of compression cords (15). Again, as shown in the FIGS. 9-10, a portion of the compression cord (15) may be secured to a portion of the socket frame (10). In this embodiment, one or more compression cords (15) may be coupled with a compression actuator (18) and further positioned within a surface channel (16) that traverses the socket frame (10) as well as at least one compression cell (1), and preferably an insert channel (13) of an adaptor support (11). In this embodiment, innervation of the compression actuator (18) may cause retraction of the compression cord (15), which in this embodiment has sufficiently elastic properties to allow it to be stretched in response to the compression actuator (18) causing the inward movement of the compression cell(s) (1).

Referring specifically to FIGS. 8-10, in one preferred embodiment a compression actuator (18) may be laterally coupled with the compression cell (5) through one or more laterally positioned compression cords (15). In this embodiment, innervation of the compression actuator (18) may cause the retraction of the compression cords (15) which may cause the coupled compression cells (1) to proximally contract generating an inward lateral compressive force, where lateral in this instance may mean approximately parallel with the residual limb thereby generating a large surface area connection when it is secured within the socket frame (10). Lateral may also include the swivel action of the disarticulated positions of the compression cells (1). Such independent swivel action may allow the disarticulated compression cells (1) to conform to the surface of a sloping residual limb thereby generating a large surface area connection.

In another embodiment, a compression actuator (18), and preferably a twist compression actuator such as a Boa® cable actuator, may be coupled with one or a plurality of coupled or disarticulated compression cells (1) through one or a plurality of compression cords (15). In this embodiment, twisting of the twist compression actuator (18) may cause the winding-up of the cord(s) (15) causing the compression cell(s) (1) to proximally contract generating an inward compressive force. This compressive force may work to secure a residual limb within the socket frame (10).

As noted above, in certain embodiments, the inventive technology may accommodate and secure a residual limb within an adaptive compression socket (22) or liner (23). In a preferred embodiment compression, lateral or otherwise, may result in the reduction of the volume of the residual limb interface. As part of the invention, compression, including lateral compression of coupled or disarticulated compression cells (1) may include, but not limited to: compression resulting in at least a 5% reduction in the volume of the residual limb interface; compression resulting in at least a 10% reduction in the volume of the residual limb interface; compression resulting in at least a 15% reduction in the volume of the residual limb interface; compression) resulting in at least a 20% reduction in the volume of the residual limb interface (10); compression resulting in at least a 25% reduction in the volume of the residual limb interface; and compression resulting in at least a 30% reduction in the volume of the residual limb interface.

In one embodiment the invention may include an improved 3-D printed adaptive compression socket (22). In one preferred embodiment, a diagnostic evaluation of a patient in need of a prosthetic device may be performed and the initial three-dimensional shape of the residual limb may be digitally generated. The digital generation of this 3-D model may be captured by invasive, or non-invasive diagnostic techniques known in the art. In a preferred embodiment, this 3-D model of the residual limb may be digitally captured and communicated to a computer system that may further process the 3-D model and generate a digital output for a customized 3-D printed adaptive compression socket (22), adaptive compression liner (23) and/or compression cells (1) configured to conform to the shape of the 3-D model of the residual limb. In this embodiment, the digital output for a customized 3-D printed disarticulated adaptive compression socket (22), adaptive compression liner (23) and/or compression cells (1) may be transmitted to a fabrication component or may be outputted into a CAD or other file format for automated mechanical or manual production. In another preferred embodiment, the computer system may upload the 3-D model and generate a digital output for a customized 3-D printed adaptive compression socket (22), adaptive compression liner (23) and/or compression cells (1) configured to conform to the shape of the 3-D model of the residual limb that may further be input into a 3-D fabrication device, such as a 3-D printer. The 3-D printing device may execute the 3-D model file and fabricate a rigid adaptive compression socket (22), adaptive compression liner (23) and/or compression cells (1) configured to conform to the shape of the 3-D model of the residual limb. Generally, a adaptive compression socket (22), adaptive compression liner (23) and/or compression cells (1) may be generated by a rapid 3-D printing/prototyping process may be made from a variety of materials, and preferably composites, generally known in the art.

As further shown in FIGS. 9-10, in this embodiment a plurality of compression cells (1) may be responsive to a single compression actuator (18) through a compression cord (15). In this embodiment, a cord cylinder (17) securing a compression cord (15) responsive to a single compression actuator (18) may be further secured within an surface channel (16) along the internal or external surface of a rigid socket frame (10) of the invention. The secured compression cord (15) may cross-over from the surface channel (16) of the rigid socket frame (10) and be secured within a first insert channel (13), which in this embodiment may pass along the length of one or more coupled or disarticulated compression cells (1).

Again, referring to FIGS. 9-10, in this embodiment, the compression cord (15), may cross-over from the first insert channel (13) of the disarticulated compression cell (5) and be secured within a surface channel (16) positioned along the internal or external surface of the socket frame (10). Again, the compression cord (15) may cross-over from the surface channel (16) positioned along the internal or external surface of the socket frame (10) and be secured within a second insert channel (13) one or more coupled or disarticulated compression cells (1). As further shown in the Figures, in this manner, a plurality of coupled or disarticulated compression cells (1) may be coupled together by a compression cord (15) and be made responsive to, in this embodiment a single compression actuator (18), such that activation of the single compression actuator (18) may generate the synchronous retraction or compression of the coupled or disarticulated compression cells (1) positioned within compression slots (19). In a preferred embodiment, such coupled or disarticulated compression cells (1) may be positioned in opposing positions, and preferably four opposing positions.

Notably, while in this embodiment the first and second insert channels (13) are positioned length wise along the coupled or disarticulated compression cells (1), in alternative embodiments first and second insert channels (13) may be positioned horizontally and operated in a similar fashion as described above to generate a synchronous retraction or compression of the coupled or disarticulated compression cells (1).

The invention may include an adaptive compression socket (22), and preferably a disarticulated 3-D printed adaptive compression socket (22) generated by a rapid 3-D printing/prototyping, configured to exhibit enhanced compression of the residual limb positioned within the frame socket (10). In this preferred embodiment, a 3-D adaptive compression socket (22) having a one or series of coupled compression cells (1) having one or more insert channels (13), which may be integral or part of an adaptor support (11), that are positioned in an off-set or staggered configuration compared to a surface channel (16) on the external or internal frame surface of a rigid socket frame (10). In this preferred embodiment, activation of the cord actuator (18) may cause the compression cord (15) to retract causing the compression cell (1) to compress against the residual limb positioned within the internal cavity of the socket frame (10), wherein the staggered configuration generates a longer transit distance of the compression cell (1) generating enhanced compression against a residual limb positioned within the internal cavity of the socket frame (10), than if the surface channels (16) and insert channels (13) were approximately aligned.

In one embodiment, the compression matrix (1) may be configured to have sufficient width that when the residual limb is positioned within the internal cavity of the socket frame (10) the compression cell(s) (1) is pushed outward forming the staggered alignment between the surface channels (16) and insert channels (13). In this embodiment, the compression cord (15) may initially be configured to be sufficiently loose to allow the expansion of the compression cell(s) (1) in response to the insertion of the residual limb into the frame socket (10). As noted above, the compression actuator (18) may be adjusted by the user to increase or decrease compression as needed.

The invention may include a thermal indicator system that incorporates one or more thermogenic compounds that may be coupled to a compression cell (1), and preferably on the contact surface (2) of the compression cell (1), and indicate the temperature of the surface of the cell (1) when placed adjacent to a residual limb. In this configuration, the temperature indicator can identify points of high and low contact engagement between the contact surface (2) of the compression cell (1) and the residual limb. Areas of high contact may indicate areas of high contact pressure which may indicate excessive of engagement between the socket frame (10), internal frame surface (24), and/or compression cell(s) (1) of the invention and the residual limb leading to discomforted and a lack of proper fit of the residual limb within the socket. Moreover, areas of low contact may indicate areas of low contact pressure which may indicate a lack of engagement between the socket frame (10), internal frame surface (24), and/or compression cell(s) (1) of the invention and the residual limb.

In a preferred embodiment, a thermal indicator system of the invention may include a thermal surface (25), which may be positioned on or impregnated onto the contact surface (2) of a compression cell (1). In this embodiment, a thermal surface (25) may include quantity of one or more thermochromic indicators that may be secured to or impregnated within a thermal surface (25) and calibrated such that the thermal energy generated from a the residual limb contacting the thermal surface (25) during use and/or fitting, may causes the thermochromic indicator(s) to transmit a temperature signal when the thermal energy reaches at least one pre-determined temperature threshold. As noted above, areas of high contact may generate more heat and as such, provide a temperature signal, such as a color indicating high, or focused contact between the contact surface (2) of a compression cell (1) and the residual limb. Conversely, areas of low contact may generate less heat and as such, provide a temperature signal, such as a color indicating low, or lack of contact between the contact surface (2) of a compression cell (1) and the residual limb. In this embodiment, the position, size and configuration of one or more compression cell (1) along a prosthetic liner may be adjusted according to a user's desired temperature indicator profile.

In another preferred embodiment, a thermal indicator system of the invention may include a thermal surface (25), which may be positioned on or impregnated onto the contact surface (2) of a prosthetic liner (6). In this embodiment, a thermal surface (25) may include quantity of one or more thermochromic indicators that may be secured to or impregnated within a thermal surface (25) and calibrated such that the thermal energy generated from a the residual limb contacting the thermal surface (25) on the prosthetic liner (6) during use and/or fitting, may causes the thermochromic indicator(s) to transmit a temperature signal when the thermal energy reaches at least one pre-determined temperature threshold.

For example, in one embodiment, the thermal surface (25) may be calibrated using a quantity of one or more thermochromic indicators that provide an acceptable or desired pressure/heat range for optimal fitting. In this embodiment, the size and position of individual compression cells (1), or the shape and fit of a prosthetic liner (6) can be configured or adjusted such that the temperature signal of the thermal surface is within a desired range. Examples of thermochromic indicators may include any substance, compound or mixture that may undergo some type or perceivable transformation in response to heat, in this case, heat being conducted from a residual limb. Examples of specific thermochromic indicators that may be used in a thermal surface (25) may include, but not be limited to: thermochromic paint, thermochromic dyes, a temperature strip, thermochromic chemicals; thermochromic strips, thermochromic pigments; and thermochromic coatings.

Naturally, all embodiments discussed herein are merely illustrative and should not be construed to limit the scope of the inventive technology consistent with the broader inventive principles disclosed. As may be easily understood from the foregoing, the basic concepts of the present inventive technology may be embodied in a variety of ways. It generally involves systems, methods, techniques as well as devices to accomplish an improved compressive cell and systems to generate an adaptive compression profile for a prosthetic device. In this application, the methods and apparatus for the aforementioned systems are disclosed as part of the results shown to be achieved by the various devices described and as steps which are inherent to utilization. They are simply the natural result of utilizing the devices as intended and described. In addition, while some devices are disclosed, it should be understood that these not only accomplish certain methods but also can be varied in a number of ways. Importantly, as to all of the foregoing, all of these facets should be understood to be encompassed by this disclosure.

While the invention has been described in connection with a preferred embodiment, it is not intended to limit the scope of the invention to the particular form set forth, but on the contrary, it is intended to cover such alternatives, modifications, and equivalents as may be included within the spirit and scope of the invention as defined by the statements of invention. As can be easily understood from the foregoing, the basic concepts of the present invention may be embodied in a variety of ways. It involves both techniques as well as devices to accomplish the appropriate system. In this application, the techniques are disclosed as part of the results shown to be achieved by the various devices described and as steps which are inherent to utilization. They are simply the natural result of utilizing the devices as intended and described. In addition, while some devices are disclosed, it should be understood that these not only accomplish certain methods but also can be varied in a number of ways. Importantly, as to all of the foregoing, all of these facets should be understood to be encompassed by this disclosure.

The discussion included in this application is intended to serve as a basic description. The reader should be aware that the specific discussion may not explicitly describe all embodiments possible; many alternatives are implicit. It also may not fully explain the generic nature of the invention and may not explicitly show how each feature or element can actually be representative of a broader function or of a great variety of alternative or equivalent elements. Again, these are implicitly included in this disclosure. Where the invention may be described in some instances in method-oriented terminology, each element of the claims corresponds to a device and vice versa. Apparatus claims may not only be included for the device described, but also method or process claims may be included to address the functions the invention and each element performs. Neither the description nor the terminology is intended to limit the scope of the claims that will be included in any subsequent patent application.

It should also be understood that a variety of changes may be made without departing from the essence of the invention. Such changes are also implicitly included in the description. They still fall within the scope of this invention. A broad disclosure encompassing the explicit embodiment(s) shown, the great variety of implicit alternative embodiments, and the broad methods or processes and the like are encompassed by this disclosure and may be relied upon when drafting any claims. It should be understood that such language changes and broader or more detailed claiming may be accomplished at a later date (such as by any required deadline) or in the event the applicant subsequently seeks a patent filing based on this filing. With this understanding, the reader should be aware that this disclosure is to be understood to support any subsequently filed patent application that may seek examination of as broad a base of claims as deemed within the applicant's right and may be designed to yield a patent covering numerous aspects of the invention both independently and as an overall system.

Further, each of the various elements of the invention and claims may also be achieved in a variety of manners. Additionally, when used or implied, an element is to be understood as encompassing individual as well as plural structures that may or may not be physically connected. This disclosure should be understood to encompass each such variation, be it a variation of an embodiment of any apparatus embodiment, a method or process embodiment, or even merely a variation of any element of these. Particularly, it should be understood that as the disclosure relates to elements of the invention, the words for each element may be expressed by equivalent apparatus terms or method terms—even if only the function or result is the same. Such equivalent, broader, or even more generic terms should be considered to be encompassed in the description of each element or action. Such terms can be substituted where desired to make explicit the implicitly broad coverage to which this invention is entitled. As but one example, it should be understood that all actions may be expressed as a means for taking that action or as an element which causes that action. Similarly, each physical element disclosed should be understood to encompass a disclosure of the action which that physical element facilitates. Regarding this last aspect, as but one example, the disclosure of a “support” should be understood to encompass disclosure of the act of “supporting”—whether explicitly discussed or not—and, conversely, were there effectively disclosure of the act of “supporting”, such a disclosure should be understood to encompass disclosure of a “supporting method and/or technique, and/or device” and even a “means for supporting.” Such changes and alternative terms are to be understood to be explicitly included in the description.

Any patents, publications, or other references mentioned in this application for patent, such as in the specification or an IDS are hereby incorporated herein by reference in their entirety. Any priority case(s) claimed by this application is hereby appended and hereby incorporated herein by reference in their entirety. In addition, as to each term used it should be understood that unless its utilization in this application is inconsistent with a broadly supporting interpretation, common dictionary definitions should be understood as incorporated for each term and all definitions, alternative terms, and synonyms such as contained in the Random House Webster's Unabridged Dictionary, second edition are hereby incorporated herein by reference in their entirety. Finally, all references listed in the list of References To Be Incorporated By Reference In Accordance With The Patent Application or other information disclosure statement and the like filed with the application are hereby appended and hereby incorporated herein by reference in their entirety, however, as to each of the above, to the extent that such information or statements incorporated by reference might be considered inconsistent with the patenting of this/these invention(s) such statements are expressly not to be considered as made by the applicant(s).

Thus, the applicant(s) should be understood to have support to claim and make a statement of invention to at least: i) each of the methods and/or apparatus for providing a compression cell and system for generating an adaptable compression profile in a prosthetic device as herein disclosed and described, ii) the related methods disclosed and described, iii) similar, equivalent, and even implicit variations of each of these devices and methods, iv) those alternative designs which accomplish each of the functions shown as are disclosed and described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such systems or components, ix) each system, method, and element shown or described as now applied to any specific field or devices mentioned, x) methods and apparatuses substantially as described hereinbefore and with reference to any of the accompanying examples, xi) the various combinations and permutations of each of the elements disclosed, xii) each potentially dependent claim or concept as a dependency on each and every one of the independent claims or concepts presented, and xiii) all inventions described herein.

With regard to claims whether now or later presented for examination, it should be understood that for practical reasons and so as to avoid great expansion of the examination burden, the applicant may at any time present only initial claims or perhaps only initial claims with only initial dependencies. The office and any third persons interested in potential scope of this or subsequent applications should understand that broader claims may be presented at a later date in this case, in a case claiming the benefit of this case, or in any continuation in spite of any preliminary amendments, other amendments, claim language, or arguments presented, thus throughout the pendency of any case there is no intention to disclaim or surrender any potential subject matter. It should be understood that if or when broader claims are presented, such may require that any relevant prior art that may have been considered at any prior time may need to be re-visited since it is possible that to the extent any amendments, claim language, or arguments presented in this or any subsequent application are considered as made to avoid such prior art, such reasons may be eliminated by later presented claims or the like. Both the examiner and any person otherwise interested in existing or later potential coverage or considering if there has at any time been any possibility of an indication of disclaimer or surrender of potential coverage, should be aware that no such surrender or disclaimer is ever intended or ever exists in this or any subsequent application. Limitations such as arose in Hakim v. Cannon Avent Group, PLC, 479 F.3d 1313 (Fed. Cir 2007), or the like are expressly not intended in this or any subsequent related matter. In addition, support should be understood to exist to the degree required under new matter laws—including but not limited to European Patent Convention Article 123(2) and United States Patent Law 35 USC 132 or other such laws—to permit the addition of any of the various dependencies or other elements presented under one independent claim or concept as dependencies or elements under any other independent claim or concept. In drafting any claims at any time whether in this application or in any subsequent application, it should also be understood that the applicant has intended to capture as full and broad a scope of coverage as legally available. To the extent that insubstantial substitutes are made, to the extent that the applicant did not in fact draft any claim so as to literally encompass any particular embodiment, and to the extent otherwise applicable, the applicant should not be understood to have in any way intended to or actually relinquished such coverage as the applicant simply may not have been able to anticipate all eventualities; one skilled in the art, should not be reasonably expected to have drafted a claim that would have literally encompassed such alternative embodiments.

Further, if or when used, the use of the transitional phrase “comprising” is used to maintain the “open-end” claims herein, according to traditional claim interpretation. Thus, unless the context requires otherwise, it should be understood that the term “comprise” or variations such as “comprises” or “comprising”, are intended to imply the inclusion of a stated element or step or group of elements or steps but not the exclusion of any other element or step or group of elements or steps. Such terms should be interpreted in their most expansive form so as to afford the applicant the broadest coverage legally permissible. It should be understood that this application also provides support for any combination of elements in the claims and even incorporates any desired proper antecedent basis for certain claim combinations such as with combinations of method, apparatus, process, and the like claims.

Any claims set forth at any time are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon. The inventive subject matter is to include, but certainly not be limited as, a system substantially as herein described with reference to any one or more of the Figures and Description (including the following: for example, the process according to any claims and further comprising any of the steps as shown in any Figures, separately, in any combination or permutation).

Finally, Applicant reserves the right to seek additional design patent protections over the claimed invention; such that the drawings are fully enabled so as to allow one of ordinary skill in the art to know that the claimed design was in Applicant's possession at the time of filing. As such, it should be noted that any broken lines are to be included for the purpose of illustrating environmental matter and form no part of the claimed design should such become necessary.

1-70. (canceled)

71. An adaptive compression prosthetic system comprising:
a compression cell having:
a compression matrix positioned between a contact surface and a support surface;
an adaptor positioned on said support surface; and
wherein the compression cell can be secured to a prosthetic liner having one or more cell interface positions generating an adaptable compression profile on a residual limb positioned in the liner.

72. The system of claim 71, and further comprising a secondary adaptor positioned on said contact surface.

73. The system of claim 71, wherein said compression matrix comprises a variably sized compression matrix.

74. The system of claim 71, wherein said compression matrix comprises a collapsible scaffold matrix.

75. The system of claim 71, wherein said compression cell comprises an integral compression cell.

76. The system of claim 75, wherein said integral compression cell comprises a 3D-printed integral compression cell.

77. The system of claim 71, wherein said adaptor comprises an extension configured to be secured in the cell interface position on the prosthetic liner.

78. The system of claim 71, wherein said adaptor comprises a coupler configured to be secured to an external surface on the prosthetic liner.

79. The system of claim 71, wherein the one or more cell interface positions comprise one or more cell interface positions on the external surface of the liner, the internal surface of the liner, or both the external and internal surfaces of the liner.

80. The system of claim 71, wherein said contact surface comprises a thermal surface.

81. An adaptive compression prosthetic system comprising:
a compression cell having:
a compression matrix positioned between a contact surface and a support surface;
an adaptor positioned on said support surface; and
wherein the compression cell can be secured to a prosthetic socket frame having one or more cell interface positions generating an adaptable compression profile on a residual limb positioned in the socket frame.

82. The system of claim 81, and further comprising a secondary adaptor positioned on said contact surface.

83. The system of claim 81, wherein said compression matrix comprises a variably sized compression matrix.

84. The system of claim 81, wherein said compression matrix comprises a collapsible scaffold matrix.

85. The system of claim 81, wherein said compression cell comprises an integral compression cell.

86. The system of claim 81, wherein said adaptor comprises an extension configured to be secured in the cell interface position on the prosthetic socket frame.

87. The system of claim 81, wherein said adaptor comprises a coupler configured to be secured to an external surface on the prosthetic socket frame.

88. The system of claim 81, wherein the one or more cell interface positions comprise one or more cell interface positions on the external surface of the socket frame, the internal surface of the socket frame, or both the external and internal surfaces of the socket frame.

89. The system of claim 81, wherein said contact surface comprises a thermal surface.

90. An adaptive compression socket comprising:
a prosthetic socket frame configured to secure a residual limb having a plurality of compression slots;
one or more compression cells coupled with an adaptor support having one or more insert channels; and
at least one compression actuator responsive to at least one compression cord secured within said insert channels, wherein said compression actuator is configured to retract said secured compression cord so as to compress said compression cell securing said residual limb within said socket frame.

91. The socket of claim 90, wherein said socket frame is selected from: a transfemoral socket frame, a transhumeral socket frame, a transradial socket frame, a transtibial socket frame, a symes socket frame, a hip disarticulation socket frame, a knee disarticulation socket frame, and a wrist disarticulation socket frame.
